(19) United States
(12) Patent Application Publication
(10) Pub. No.: US 2026/0286340 A1
(43) Pub. Date: Sep. 24, 2026

(54) BIOLOGICAL MATERIAL PURIFICATION METHOD AND MAGNETIC SEPARATION DEVICE

(71) Applicant: SEIKO EPSON CORPORATION, TOKYO (JP)

(72) Inventors: Soichi MORIYA, SUWA-SHI (JP); Naoyuki TOYODA, SUWA-SHI (JP)

(21) Appl. No.: 19/571,572

(22) Filed: Mar. 19, 2026

(30) Foreign Application Priority Data
Mar. 19, 2025 (JP) 2025-044582

(51) Int. Cl. C12N 15/10; B03C 1/01; B03C 1/28; B03C 1/30

(52) U.S. Cl. CPC C12N 15/1013; B03C 1/01; B03C 1/288; B03C 1/30; B03C 2201/18; B03C 2201/26

(57) ABSTRACT

Provided is a biological material purification method, which is a method for purifying a biological material. The method includes: an adsorption step of mixing magnetic beads and the biological material and adsorbing the biological material to the magnetic beads in the obtained mixed liquid; a separation step of separating, from the mixed liquid, the magnetic beads to which the biological material is adsorbed; and an elution step of eluting the biological material from the magnetic beads. The magnetic beads contain a magnetic metal, and a heat generation treatment of causing the magnetic beads to generate heat by induction heating is performed.

The present application is based on, and claims priority from JP Application Serial Number 2025-044582, filed March 19, 2025, the disclosure of which is hereby incorporated by reference herein in its entirety.

### BACKGROUND

### 1. Technical Field

The present disclosure relates to a biological material purification method and a magnetic separation device.

### 2. Related Art

JP-A-2017-176023 discloses a nucleic acid-binding solid-phase carrier including magnetic particles of an amorphous alloy containing Fe, Cr, Si, and B, and a silicon oxide film provided on surfaces of the magnetic particles.

JP-A-2017-176023 discloses a nucleic acid extraction method including a step of adsorbing a nucleic acid to a nucleic acid-binding solid-phase carrier in an adsorption liquid containing a chaotropic substance, a step of cleaning the nucleic acid-binding solid-phase carrier by disposing, in a wash solution, the nucleic acid-binding solid-phase carrier to which the nucleic acid is adsorbed and vibrating, by a magnetic force, the nucleic acid-binding solid-phase carrier to which the nucleic acid is adsorbed, and a step of eluting the nucleic acid into an elution liquid by disposing, in the elution liquid, the nucleic acid-binding solid-phase carrier to which the nucleic acid is adsorbed.

According to the nucleic acid extraction method described in JP-A-2017-176023, the nucleic acid-binding solid-phase carrier to which the nucleic acid is adsorbed can be moved by application of an external magnetic field and cleaned, so that the nucleic acid can be efficiently extracted.

JP-A-2017-176023 is an example of the related art.

However, the nucleic acid extraction method described in JP-A-2017-176023 has a problem in that extraction efficiency of a nucleic acid is low because each of the step of adsorbing the nucleic acid to the carrier, the step of cleaning the carrier, and the step of eluting the nucleic acid takes time.

### SUMMARY

A biological material purification method according to an application example of the present disclosure is a method for purifying a biological material. The method includes:

an adsorption step of mixing magnetic beads and the biological material and adsorbing the biological material to the magnetic beads in the obtained mixed liquid;

a separation step of separating, from the mixed liquid, the magnetic beads to which the biological material is adsorbed; and

an elution step of eluting the biological material from the magnetic beads. The magnetic beads contain a magnetic metal, and a heat generation treatment of causing the magnetic beads to generate heat by induction heating is performed.

A magnetic separation device according to an application example of the present disclosure includes:

a container holding unit configured to hold a container that accommodates magnetic beads and that extends along an axis coupling a bottom portion and an opening portion; and

an electromagnetic coil disposed outside a side portion coupling the bottom portion and the opening portion.

### BRIEF DESCRIPTION OF THE DRAWINGS

FIG. 1 is a flowchart illustrating a configuration of a biological material purification method according to an embodiment.

FIG. 2 is a schematic diagram illustrating the biological material purification method illustrated in FIG. 1.

FIG. 3 is a schematic diagram illustrating the biological material purification method illustrated in FIG. 1.

FIG. 4 is a schematic diagram illustrating the biological material purification method illustrated in FIG. 1.

FIG. 5 is a perspective view illustrating an example of an induction heating device used for the biological material purification method according to the embodiment.

FIG. 6 is a cross-sectional view illustrating an example of the induction heating device used for the biological material purification method according to the embodiment.

FIG. 7 is a cross-sectional view illustrating a configuration of a magnetic separation device according to the embodiment.

FIG. 8 is a cross-sectional view illustrating a configuration of the magnetic separation device according to the embodiment.

FIG. 9 is a cross-sectional view illustrating a magnetic bead according to the embodiment.

FIG. 10 is Table 1 illustrating conditions of nucleic acid purification and evaluation results of nucleic acid purification using the biological material purification method of Examples and Comparative Examples.

FIG. 11 is Table 2 illustrating conditions of nucleic acid purification and evaluation results of nucleic acid purification using the biological material purification method of Examples and Comparative Examples.

FIG. 12 is Table 3 illustrating conditions of nucleic acid purification and evaluation results of nucleic acid purification using the biological material purification method of Examples and Comparative Examples.

### DESCRIPTION OF EMBODIMENTS

Hereinafter, preferred embodiments of a biological material purification method and a magnetic separation device according to the present disclosure will be described in detail with reference to the accompanying drawings.

### 1. Biological Material Purification Method

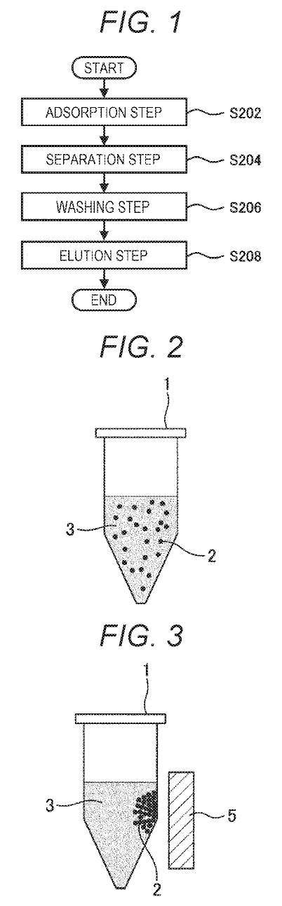

FIG. 1 is a flowchart illustrating a configuration of a biological material purification method according to an embodiment. FIGS. 2 to 4 are schematic diagrams illustrating the biological material purification method illustrated in FIG. 1.

### 1.1. Overview

The biological material purification method illustrated in FIG. 1 is a method for purifying a biological material, and includes an adsorption step S202, a separation step S204, a washing step S206, and an elution step S208.

In the adsorption step S202, a mixed liquid obtained by mixing magnetic beads 2, a biological material (not illustrated), and a lysis-adsorption liquid (liquid 3 illustrated in FIG. 2) is prepared in a container 1 illustrated in FIG. 2. Then, the biological material is adsorbed to the magnetic beads 2 in the mixed liquid.

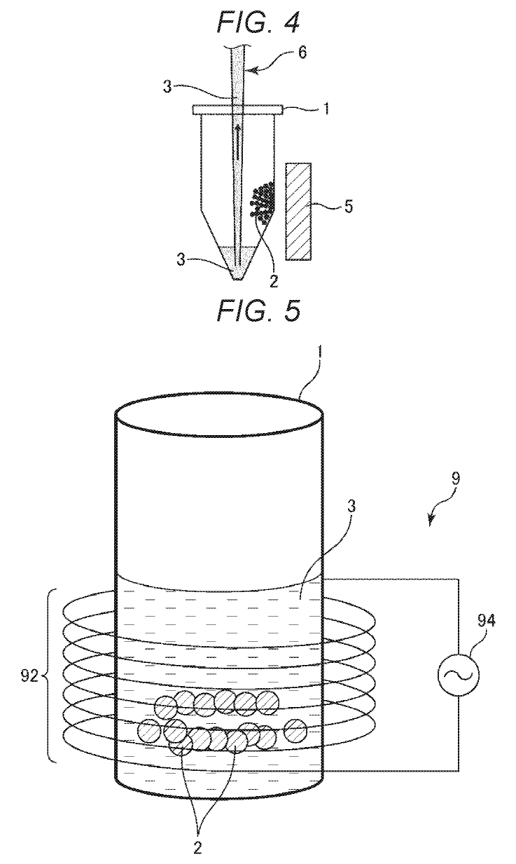

In the separation step S204, the magnetic beads 2 to which the biological material is adsorbed are magnetically separated from the mixed liquid in the container 1 as illustrated in FIGS. 3 and 4.

In the washing step S206, the magnetic beads 2 to which the biological material is adsorbed are cleaned with a wash solution in the container 1. In the present disclosure, the washing step S206 is not essential and may be omitted.

In the elution step S208, the biological material is eluted from the magnetic beads 2 to which the biological material is adsorbed into an elution liquid in the container 1.

The above magnetic bead 2 contains a magnetic metal having magnetism.

The biological material purification method according to the embodiment includes a heat generation treatment of causing the magnetic beads 2 to generate heat by induction heating in any of the biological material purification processes including the above steps.

According to such a configuration, the magnetic beads 2 can generate heat in a short time. As a result, the biological material can be efficiently purified. Specifically, the heat generation treatment of the magnetic beads 2 by induction heating is applied in at least one purification treatment of the adsorption of the biological material in the adsorption step S202, the cleaning of the magnetic beads 2 in the washing step S206, and the elution of the biological material in the elution step S208. Accordingly, the efficiency of each purification treatment can be increased.

The above magnetic bead 2 is a particulate group that captures a biological material and is used for magnetic separation. Magnetic separation is a technique of applying an external magnetic field to a container in which a solid phase containing the magnetic beads 2 and a liquid phase containing a solvent or the like are charged to magnetically attract the solid phase and thus separating the solid phase from the liquid phase.

Examples of the biological material include nucleic acids such as DNA, RNA, and hybrids thereof. The biological material may be a biological component other than the nucleic acid. The nucleic acid or the biological component may be present in a state of being contained in, for example, a biological sample such as a cell or a biological tissue, a virus, or a bacterium. In the following description, a case where the biological material is a nucleic acid will be described as an example, and the following description can be similarly applied to a case where the biological material is a biological component other than a nucleic acid.

### 1.2. Induction Heating Device

First, an example of an induction heating device used for the biological material purification method according to the embodiment will be described.

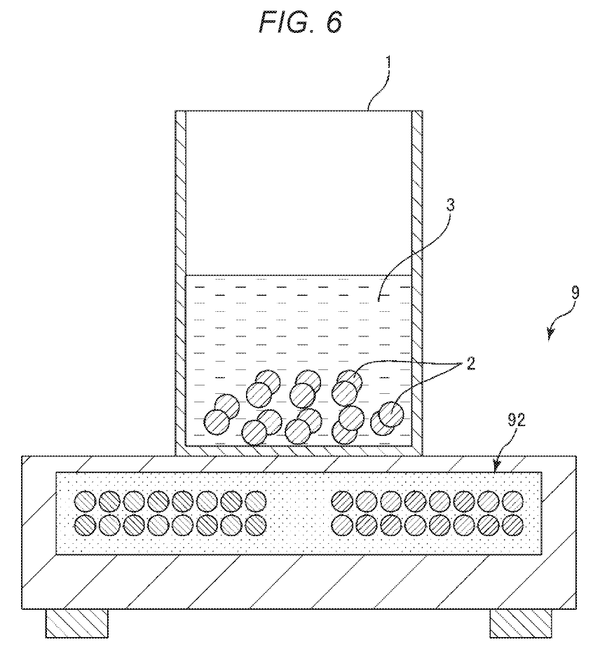

FIG. 5 is a perspective view illustrating an example of an induction heating device 9 used for the biological material purification method according to the embodiment. FIG. 6 is a cross-sectional view illustrating an example of the induction heating device 9 used for the biological material purification method according to the embodiment.

The induction heating device 9 illustrated in FIG. 5 includes an electromagnetic coil 92. The electromagnetic coil 92 is coupled to an alternating current power supply 94. The induction heating device 9 illustrated in FIG. 5 causes the magnetic beads 2 accommodated in the container 1 to generate heat by induction heating.

The electromagnetic coil 92 illustrated in FIG. 5 has a cylindrical shape as an example. Accordingly, a space that allows the container 1 to be inserted can be formed inside the electromagnetic coil 92. A constituent material of the electromagnetic coil 92 is not particularly limited, and examples thereof include copper, a copper alloy, aluminum, and an aluminum alloy.

The alternating current power supply 94 is a power supply that supplies an alternating current to the electromagnetic coil 92. When an alternating current is applied to the electromagnetic coil 92, an alternating magnetic field that changes with time is generated around the electromagnetic coil 92. The alternating magnetic field is a magnetic field whose orientation changes periodically. With this alternating magnetic field, an electromotive force is generated in the magnetic bead 2, and an eddy current is generated. The eddy current generates Joule heat, and the magnetic bead 2 generates heat. The amount of heat generated is large in the heat generation, and therefore, the temperature of the magnetic beads 2 can be increased in a short time. Accordingly, the temperature of the liquid 3 around the magnetic beads 2 also rises in a short time. As a result, the efficiency of each purification treatment described above can be increased.

The electromagnetic coil 92 illustrated in FIG. 5 is disposed in a manner of surrounding the container 1, and therefore, a strong alternating magnetic field is easily generated in the container 1. Therefore, according to the induction heating device 9 illustrated in FIG. 5, the magnetic beads 2 can be caused to efficiently generate heat.

The induction heating device 9 illustrated in FIG. 6 is the same as the induction heating device 9 illustrated in FIG. 5 except that a shape of the electromagnetic coil 92 is different.

The electromagnetic coil 92 illustrated in FIG. 6 has, for example, a flat plate shape. That is, the electromagnetic coil 92 illustrated in FIG. 5 forms such a shape obtained by crushing the cylindrical electromagnetic coil 92 illustrated in FIG. 5. Accordingly, in the induction heating device 9 illustrated in FIG. 6, the container 1 can be placed on the electromagnetic coil 92 instead of being inserted inside the electromagnetic coil 92. As a result, restrictions on the shape of the container 1 can be reduced. The magnetic beads 2 easily settle over time, and therefore, the distance between the electromagnetic coil 92 and the magnetic beads 2 can be easily reduced in the arrangement form of the container 1 illustrated in FIG. 6. Accordingly, a rate of temperature rise of the magnetic beads 2 is further increased.

### 1.3. Magnetic Separation Device

Next, a magnetic separation device according to the embodiment will be described.

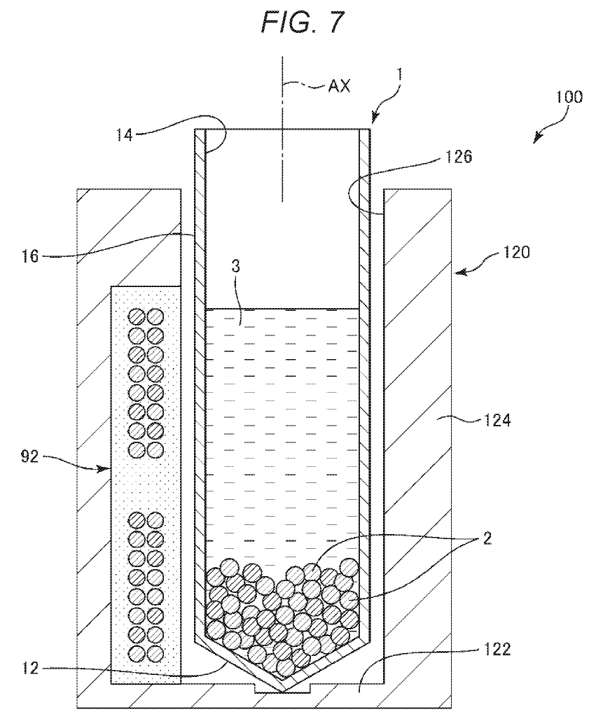

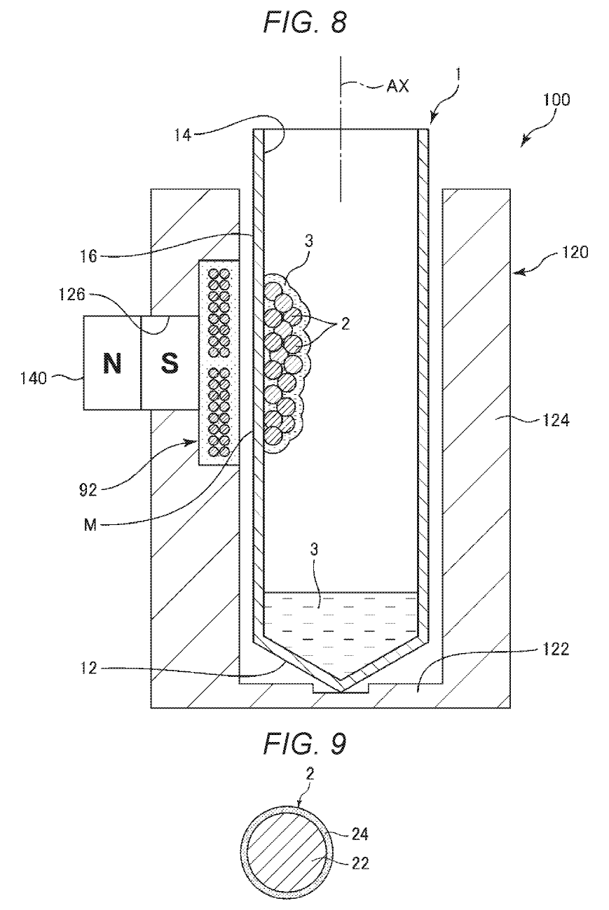

FIGS. 7 and 8 are cross-sectional views each illustrating a configuration of a magnetic separation device 100 according to the embodiment.

Hereinafter, the magnetic separation device 100 will be described, but in the following description, differences from the induction heating device 9 described above will be mainly described, and description of similar matters will be omitted. Note that, in FIGS. 7 and 8, substantially the same configurations as those illustrated in FIGS. 5 or FIG. 6 are denoted by the same reference symbols.

The magnetic separation device 100 illustrated in FIG. 7 includes a container holding unit 120 for holding the container 1, and the electromagnetic coil 92. The container holding unit 120 holds the container 1 extending along an axis AX illustrated in FIG. 7. The electromagnetic coil 92 illustrated in FIG. 7 is fixed to the container holding unit 120 and is disposed outside a side portion 16 of the container 1 held by the container holding unit 120.

According to such a configuration, the magnetic separation device 100 capable of generating an alternating magnetic field from an outside of the side portion 16 with respect to the container 1 held by the container holding unit 120 is obtained. In such a magnetic separation device 100, an alternating magnetic field can be generated over a wide range in an extending direction of the container 1 (the direction in which the axis AX extends). Accordingly, even when the magnetic beads 2 are widely dispersed in the container 1, the magnetic beads 2 can efficiently generate heat.

The magnetic beads 2 can be caused to generate heat by generating an alternating magnetic field from the outside of the side portion 16, and therefore, the magnetic beads 2 can be caused to generate heat in a state in which the magnetic beads 2 are immobilized to an inner wall of the side portion 16 and a distance is taken from a bottom of the container 1. That is, the magnetic separation device 100 is a device that enables not only the heat generation treatment of the magnetic beads 2 but also the magnetic separation treatment when a magnetic field application mechanism (not illustrated) that enables the magnetic separation treatment is added to the induction heating device 9 described above. According to such a magnetic separation device 100, the magnetic beads 2 can generate heat even during the magnetic separation treatment. Accordingly, for example, it is possible to dry the wash solution adhering to the immobilized magnetic beads 2 or to cause bumping to spatter off the elution liquid adhering to the elution liquid adhering to the immobilized magnetic beads 2.

The container 1 illustrated in FIG. 7 has a bottomed cylindrical shape extending along the axis AX. The container 1 includes a bottom portion 12, an opening portion 14, and the side portion 16 coupling the bottom portion 12 and the opening portion 14. The axis AX extends so as to couple the bottom portion 12 and the opening portion 14.

The container holding unit 120 has a function of holding the container 1. The container holding unit 120 includes a bottom portion support 122 and a side portion support 124. The bottom portion support 122 and the side portion support 124 define an insertion hole 126 into which the container 1 is to be inserted.

The bottom portion support 122 supports the bottom portion 12 of the container 1 inserted into the insertion hole 126 from below. The side portion support 124 supports the side portion 16 of the container 1 inserted into the insertion hole 126 from the outside.

The configuration of the magnetic separation device 100 is not limited to the above. For example, the container holding unit 120 may hold the container 1 with a structure other than the insertion hole 126.

The magnetic separation device 100 illustrated in FIG. 8 includes the container holding unit 120, the electromagnetic coil 92, and a magnet 140.

The container holding unit 120 and the electromagnetic coil 92 illustrated in FIG. 8 are the same as the container holding unit 120 and the electromagnetic coil 92 illustrated in FIG. 7.

The magnet 140 illustrated in FIG. 8 is disposed on a side opposite to the side portion 16 of the container 1 (behind the electromagnetic coil 92) via the electromagnetic coil 92. The magnet 140 generates a magnetic field having a constant orientation (constant magnetic field) from behind the electromagnetic coil 92 toward the container 1. Accordingly, the magnetic beads 2 can be immobilized to the inner wall of the side portion 16. That is, the magnet 140 is an example of a magnetic field application mechanism that enables the magnetic separation treatment.

According to such a configuration, the magnetic beads 2 can be efficiently caused to generate heat while immobilizing the magnetic beads 2 to the inner wall of the side portion 16. In addition, since the magnet 140 is disposed behind the electromagnetic coil 92, it is easy to immobilize the magnetic beads 2 in a vicinity of the electromagnetic coil 92. Therefore, the immobilized magnetic beads 2 can more efficiently generate heat.

The magnet 140 may be an electromagnet, and is preferably a permanent magnet. Accordingly, a power supply is not required to generate a constant magnetic field, and a reduction in size and weight of the magnetic separation device 100 is facilitated.

Examples of the permanent magnet include a neodymium iron boron magnet, a samarium-cobalt magnet, a ferrite magnet, and an alnico magnet. These can generate a sufficient constant magnetic field with a smaller size.

A magnetic flux density on a surface of the magnet 140 is preferably 50 mT or more and more preferably 200 mT or more. Accordingly, a movement speed of the magnetic beads 2 in the magnetic separation can be increased, and the immobilized magnetic beads 2 can be prevented from falling off. The surface magnetic flux density of the magnet 140 is measured by, for example, a Gauss meter using a Hall element.

In the magnetic separation device 100 illustrated in FIG. 8, a position of the electromagnetic coil 92 is not particularly limited as long as it is a position corresponding to the side portion 16 of the container 1, and is preferably a position closer to the opening portion 14 than an intermediate point M of the length of the container 1 (the length from the bottom portion 12 to the opening portion 14). The position of the electromagnetic coil 92 refers to a position of a central portion of the electromagnetic coil 92 in the extending direction of the axis AX. Therefore, the position of the central portion of the electromagnetic coil 92 is preferably located closer to the opening portion 14 than the intermediate point M. As a result, even when the liquid 3 remains on the bottom of the container 1 as illustrated in FIG. 4 after the liquid discharging treatment is performed, the contact between the fixed magnetic beads 2 and the liquid 3 can be prevented. In addition, even when the liquid 3 adhering to the immobilized magnetic beads 2 falls to the bottom of the container 1 after the liquid discharging treatment, the contact between the fixed magnetic beads 2 and the falling liquid 3 can be prevented.

The container holding unit 120 illustrated in FIG. 8 has the insertion hole 126 into which the magnet 140 can be inserted. The magnet 140 can be disposed at the above position by inserting the magnet 140 into the insertion hole 126. In this state, the magnetic flux formed by the magnet 140 acts on the magnetic beads 2, and the magnetic beads 2 can be immobilized to the inner wall of the side portion 16 of the container 1. On the other hand, it is preferable that the magnet 140 can be inserted into and removed from the insertion hole 126. Accordingly, the magnetic flux density formed in the container 1 can be reduced by removing the magnet 140 from the insertion hole 126 or moving the magnet 140 away from the container 1. As a result, the immobilization of the magnetic beads 2 can be easily released.

The container holding unit 120 may have a configuration in place of the insertion hole 126. As a configuration thereof, for example, the magnetic flux density formed in the container 1 can be changed by moving the magnet 140.

### 1.4. Adsorption Step

In the adsorption step S202, the lysis-adsorption liquid (liquid 3) to which a specimen sample containing nucleic acid is added, and the magnetic beads 2 are charged into the container 1 illustrated in FIG. 2. Then, substances accommodated in the container 1 are mixed. Accordingly, as illustrated in FIG. 2, the magnetic beads 2 are dispersed in the lysis-adsorption liquid in the container 1. The nucleic acid is usually encapsulated in an outer shell such as a cell membrane or a nucleus. Therefore, the nucleic acid is extracted from a specimen sample when the outer shell is lysed and removed by the lysis action of the lysis-adsorption liquid. Thereafter, the nucleic acid is adsorbed to the magnetic beads 2 by an adsorption action of the lysis-adsorption liquid.

As the lysis-adsorption liquid, for example, a liquid containing Proteinase K, a chaotropic substance, and the like is used. Proteinase K degrades proteins and solubilizes nucleic acids. The chaotropic substance generates chaotropic ions in an aqueous solution to reduce an interaction between water molecules, thereby destabilizing a structure. This contributes to the adsorption of nucleic acids.

As the lysis-adsorption liquid, water such as sterile water is used. Any additive may be added to the lysis-adsorption liquid. Examples of the additive include a salt, a buffer, a surfactant, an organic solvent, an acid, and a base.

Examples of the salt include sodium chloride, lithium chloride, potassium carbonate, and trisodium citrate.

Examples of the buffer include an acetate buffer, a phosphate buffer, a formic acid buffer, a citrate buffer, and a tartrate buffer.

Examples of the surfactant include a triton surfactant such as Triton X-100, a nonionic surfactant such as a Tween surfactant such as Tween20, and an anionic surfactant such as sodium N-lauroylsarcosinate (SDS).

Examples of the organic solvent include alcohols such as ethanol.

In the adsorption step S202, a heat generation treatment of causing the magnetic beads 2 to generate heat by induction heating may be performed. The lysis-adsorption liquid around the magnetic beads 2 can be heated in a short time by performing the heat generation treatment. The adsorption efficiency of nucleic acid can be increased by heating the magnetic beads 2 and the lysis-adsorption liquid around the magnetic beads 2. Accordingly, a high-purity nucleic acid can be finally purified in a high yield. The foreign substances can be decomposed by heating the magnetic beads 2 and the lysis-adsorption liquid around the magnetic beads 2. Accordingly, the foreign substances are less likely to be captured by the magnetic beads 2. Even if foreign substances are co-captured, the influence of the foreign substances can be reduced when the nucleic acid is finally tested. When the nucleic acid contained in the specimen sample is encapsulated in the outer shell, the nucleic acid can be efficiently extracted from the outer shell by heating the mixed liquid by a heat generation treatment.

When the induction heating of the magnetic beads 2 is performed in another step, the lysis-adsorption liquid may be heated by a heating method other than the induction heating in the present step. Even in this case, the efficiency of purification by heating can be improved in the entire biological material purification method. Examples of other heating methods include resistance heating and radiation heating.

The temperature of the lysis-adsorption liquid associated with the heat generation treatment of the magnetic beads 2 is not particularly limited, and is preferably 30°C or higher and 80°C or lower, more preferably 40°C or higher and 70°C or lower, and still more preferably 50°C or higher and 60°C or lower. When the temperature of the lysis-adsorption liquid is within the above range, the adsorption efficiency of the nucleic acid can be particularly increased. The thermal decomposition of the foreign substances is facilitated.

When the temperature of the lysis-adsorption liquid is lower than the above lower limit, the adsorption efficiency of nucleic acid may not be sufficiently increased. The foreign substances may not be sufficiently decomposed. On the other hand, when the temperature of the lysis-adsorption liquid is higher than the above upper limit, thermal denaturation may occur in the nucleic acid or the lysis-adsorption liquid.

The time (heating time) for which the temperature of the lysis-adsorption liquid is maintained within the above range is preferably 1 minute or longer and 60 minutes or shorter, more preferably 3 minutes or longer and 30 minutes or shorter, and still more preferably 5 minutes or longer and 15 minutes or shorter. When the heating time is within the above range, thermal denaturation associated with long-term heating can be prevented while sufficiently increasing the nucleic acid adsorption efficiency and sufficiently decomposing foreign substances.

The induction heating device 9 and the magnetic separation device 100 described above are used for the heat generation treatment of the magnetic beads 2. Each of them includes the electromagnetic coil 92 through which an alternating current flows. An alternating magnetic field can be applied to the magnetic beads 2, and the magnetic beads 2 can be inductively heated by using the electromagnetic coil 92.

The frequency of the induction heating, that is, the frequency of the alternating current flowing through the electromagnetic coil 92 is not particularly limited, and is preferably 20 kHz or more and 100 kHz or less. When the frequency is within this range, the depth of the skin effect can be optimized according to the particle diameter of the magnetic bead 2, and therefore, the heat generation treatment can be particularly efficiently performed.

When the frequency of the induction heating is less than the above lower limit or more than the upper limit, the heat generation amount may decrease.

FIG. 9 is a cross-sectional view illustrating the magnetic bead 2 according to the embodiment.

The magnetic bead 2 illustrated in FIG. 9 includes the magnetic metal particle 22 and a coating layer 24. A metal powder having magnetism is used as the magnetic metal particle 22. The coating layer 24 contains an inorganic oxide. The magnetic beads 2 will be described in detail later.

### 1.5. Separation Step

In the separation step S204, an external magnetic field is applied to the mixed liquid containing the magnetic beads 2 to which the nucleic acid is adsorbed, and the magnetic beads 2 are magnetically attracted to the inner wall of the container 1. Accordingly, as illustrated in FIG. 3, the magnetic beads 2 are moved to an inner wall of the container 1 and immobilized. As a result, the magnetic beads 2 as a solid phase and the lysis-adsorption liquid (liquid 3) as a liquid phase can be magnetically separated. In the present specification, the treatment of magnetic separation is referred to as a "magnetic separation treatment".

Before the magnetic separation treatment is performed, the substances accommodated in the container 1 are stirred as necessary. Accordingly, the probability that the nucleic acid is captured by the magnetic beads 2 can be increased. In the stirring, for example, a vortex mixer, hand shaking, or pipetting is used. This stirring is preferably performed in a state in which no external magnetic field is applied. Accordingly, the magnetic beads 2 are favorably dispersed in the lysis-adsorption liquid, and therefore, the adsorption efficiency can be increased.

In the application of the external magnetic field, for example, a magnet 5 disposed beside the container 1 is used. The magnet 5 may be an electromagnet or a permanent magnet. When the external magnetic field acts on the magnetic beads 2, the magnetic beads 2 move toward the magnet 5.

In a state in which the magnetic beads 2 are immobilized to the inner wall of the container 1 by performing the magnetic separation treatment, as illustrated in FIG. 4, the lysis-adsorption liquid (liquid 3) accumulated at the bottom of the container 1 is sucked and discharged by, for example, a pipette 6 or the like. In the present specification, the treatment of discharging the liquid 3 illustrated in FIG. 4 is referred to as a "liquid discharging treatment". With the liquid discharging treatment, the magnetic beads 2 capturing the nucleic acid remain in the container 1.

After the liquid discharging treatment, an acceleration may be applied to the container 1 as necessary. Accordingly, the lysis-adsorption liquid adhering to the magnetic beads 2 can be shaken off, and thus the unseparated lysis-adsorption liquid can be reduced. The acceleration may be a centrifugal acceleration. A centrifugal separator may be used to apply the centrifugal acceleration.

### 1.6. Washing Step

In the washing step S206, the magnetic beads 2 to which the nucleic acids are adsorbed are cleaned. Cleaning refers to a treatment of transferring foreign substances captured by the magnetic beads 2 to the wash solution and removing the foreign substances by bringing the magnetic beads 2, to which the nucleic acid is adsorbed, into contact with a wash solution and then separating the magnetic beads 2 from the wash solution again in order to remove the impurities.

Specifically, after the wash solution is charged into the container 1 subjected to the separation step S204, the magnetic separation treatment and the liquid discharging treatment described above are performed again. In this case, the liquid 3 in FIGS. 2 to 4 is a wash solution.

In the magnetic separation treatment, first, the magnetic beads 2 and the wash solution in the container 1 are stirred. Accordingly, the magnetic beads 2 are brought into contact with the wash solution and cleaned. At this time, the application of the external magnetic field may be temporarily turned off. Accordingly, the magnetic beads 2 are re-dispersed in the wash solution, and therefore, the cleaning efficiency can be increased. Thereafter, an external magnetic field is applied to immobilize the magnetic beads 2 to the inner wall of the container 1.

In the liquid discharging treatment, the wash solution accumulated on the bottom of the container 1 is discharged in a state in which the magnetic beads 2 are immobilized to the inner wall of the container 1. Accordingly, the magnetic beads 2 are cleaned.

The supply and discharging of the wash solution may be repeated twice or more in total. That is, cleaning may be performed a plurality of times. Accordingly, the foreign substances can be accurately removed.

When the cleaning is performed a plurality of times, the components in the wash solution may be different each time.

The wash solution is not particularly limited as long as the wash solution is a liquid that does not promote elution of the nucleic acid and does not promote binding of the foreign substance to the magnetic bead 2, and examples thereof include organic solvents such as ethanol, isopropyl alcohol, and acetone, aqueous solutions thereof, and low-salt-concentration aqueous solutions, in addition to water.

Various additives may be added to the wash solution. Examples of the additives include buffers such as an acetic acid buffer and a phosphoric acid buffer, salts such as sodium chloride, and surfactants.

In the washing step S206, a heat generation treatment of causing the magnetic beads 2 to generate heat by induction heating may be performed. The wash solution around the magnetic beads 2 can be heated in a short time by performing the heat generation treatment. The cleaning efficiency of the magnetic beads 2 can be increased by heating the magnetic beads 2 and the wash solution around the magnetic beads 2. Accordingly, a high-purity nucleic acid can be finally purified in a high yield.

When the induction heating of the magnetic beads 2 is performed in another step, the wash solution may be heated by a heating method other than the induction heating in the present step.

The temperature of the wash solution associated with the heat generation treatment of the magnetic beads 2 is not particularly limited, and is preferably 30°C or higher and 80°C or lower, more preferably 40°C or higher and 70°C or lower, and still more preferably 50°C or higher and 60°C or lower. When the temperature of the wash solution is within the above range, the cleaning efficiency of the magnetic beads 2 can be particularly increased.

When the temperature of the wash solution is lower than the lower limit, the cleaning efficiency of the magnetic beads 2 may not be sufficiently increased. On the other hand, when the temperature of the wash solution is higher than the above upper limit, thermal denaturation may occur in the nucleic acid or the wash solution.

The time (heating time) for which the cleaning temperature is maintained within the above range is preferably 1 minute or longer and 60 minutes or shorter, more preferably 3 minutes or longer and 30 minutes or shorter, and still more preferably 5 minutes or longer and 15 minutes or shorter. When the heating time is within the above range, the cleaning efficiency of the magnetic beads 2 can be sufficiently increased, and thermal denaturation associated with long-term heating can be prevented.

The induction heating device 9 and the magnetic separation device 100 described above are used for the heat generation treatment of the magnetic beads 2. Each of them includes the electromagnetic coil 92 through which an alternating current flows. An alternating magnetic field can be applied to the magnetic beads 2, and the magnetic beads 2 can be inductively heated by using the electromagnetic coil 92.

The frequency of the induction heating, that is, the frequency of the alternating current flowing through the electromagnetic coil 92 is the same as that in the adsorption step S202.

In the washing step S206, the wash solution adhering to the magnetic beads 2 may be dried after the cleaning is completed. The drying of the wash solution may be performed by natural drying, or may be performed using heat generation of the magnetic beads 2 by induction heating. The latter can be performed using the magnetic separation device 100. That is, after the magnetic beads 2 are immobilized to the inner wall of the side portion 16 of the container 1 and the liquid discharging treatment is performed as illustrated in FIG. 4, a drying treatment of heating the magnetic beads 2 and drying the wash solution is performed. Accordingly, a small amount of the wash solution adhering to the magnetic beads 2 can be vaporized and removed in a short time. As a result, the wash solution (residual liquid) adhering to the magnetic beads 2 can be prevented from being brought into the elution step S208. Accordingly, when the nucleic acid after purification is tested, the occurrence of test failure associated with the residual liquid can be prevented.

When the wash solution is dried, the temperature of the wash solution associated with the heat generation treatment of the magnetic beads 2 may be set within the above range, or may be equal to or higher than the boiling point of the wash solution. In the latter case, the wash solution can be dried in a shorter time.

The washing step S206 may be performed as necessary and may be omitted when cleaning is not necessary. Only one or both of the heating treatment of the wash solution and the drying treatment of the wash solution included in the washing step S206 may be performed.

### 1.7. Elution Step

In the elution step S208, the nucleic acids captured by the magnetic beads 2 are eluted into the elution liquid. The elution is a treatment of transferring the nucleic acid to the elution liquid by bringing the magnetic beads 2 to which the nucleic acid is adsorbed into contact with the elution liquid.

Specifically, after the elution liquid is charged into the container 1 containing the magnetic beads 2 to which the nucleic acids are adsorbed, the magnetic separation treatment and the liquid discharging treatment described above are performed again. In this case, the liquid 3 in FIGS. 2 to 4 is an elution liquid.

In the magnetic separation treatment, first, the magnetic beads 2 and the elution liquid in the container 1 are stirred. Accordingly, the elution liquid comes into contact with the magnetic bead 2, and the nucleic acid is eluted into the elution liquid. At this time, the application of the external magnetic field may be temporarily turned off. Accordingly, the magnetic beads 2 are re-dispersed in the elution liquid, and therefore, the elution efficiency can be increased. Thereafter, an external magnetic field is applied to immobilize the magnetic beads 2 to the inner wall of the container 1.

In the liquid discharging treatment, the elution liquid accumulated on the bottom of the container 1 is discharged in a state in which the magnetic beads 2 are immobilized to the inner wall of the container 1. Accordingly, the elution liquid containing the nucleic acid can be collected.

The elution liquid is not particularly limited as long as it is a liquid that promotes elution of the nucleic acid captured by the magnetic beads 2, and examples thereof include water such as sterile water and pure water.

Any additive may be added to the elution liquid. Examples of the additive include a salt, a buffer, a surfactant, an organic solvent, an acid, and a base.

In the elution step S208, a heat generation treatment of causing the magnetic beads 2 to generate heat by induction heating may be performed. The elution liquid around the magnetic beads 2 can be heated in a short time by performing the heat generation treatment. The elution efficiency of the nucleic acid can be increased by heating the magnetic beads 2 and the elution liquid around the magnetic beads 2. Accordingly, a high-purity nucleic acid can be finally purified in a high yield.

The elution step S208 may include a treatment of heating the elution liquid by a heat generation treatment to promote vaporization of the elution liquid and concentrate the elution liquid. Accordingly, the concentration of the elution liquid can be increased, and the nucleic acid purification can be efficiently performed. Accordingly, when nucleic acid detection using, for example, real-time PCR is performed on the purified product, there is an advantage that fewer PCR cycles are required to reach a detectable level of an amplified product.

When the induction heating of the magnetic beads 2 is performed in another step, the elution liquid may be heated by a heating method other than the induction heating in the present step.

The temperature of the elution liquid associated with the heat generation treatment of the magnetic beads 2 is not particularly limited, and is preferably 30°C or higher and 80°C or lower, more preferably 40°C or higher and 70°C or lower, and still more preferably 50°C or higher and 60°C or lower. When the temperature of the elution liquid is within the above range, the elution efficiency of the nucleic acid can be particularly increased.

When the temperature of the elution liquid is lower than the lower limit, the elution efficiency of the nucleic acid may not be sufficiently increased. On the other hand, when the temperature of the elution liquid is higher than the upper limit, thermal denaturation may occur in the nucleic acid or the elution liquid.

The time (heating time) for which the temperature of the elution liquid is maintained within the above range is preferably 1 minute or longer and 60 minutes or shorter, more preferably 3 minutes or longer and 30 minutes or shorter, and still more preferably 5 minutes or longer and 15 minutes or shorter. When the heating time is within the above range, thermal denaturation associated with long-term heating can be prevented while sufficiently increasing the nucleic acid elution efficiency.

The induction heating device 9 and the magnetic separation device 100 described above are used for the heat generation treatment of the magnetic beads 2. Each of them includes the electromagnetic coil 92, and therefore, the magnetic beads 2 can be inductively heated.

The frequency of the induction heating, that is, the frequency of the alternating current flowing through the electromagnetic coil 92 is the same as that in the adsorption step S202.

The elution step S208 may include a treatment of shaking off, without being completely separated, the elution liquid (residual liquid) adhering to the magnetic beads 2 to the bottom of the container 1 after performing the liquid discharging treatment once. The residual elution liquid can be shaken off by utilizing the heat generation of the magnetic beads 2 generated by the induction heating. Specifically, after the liquid discharging treatment is performed, the heat generation treatment is performed. The amount of the residual liquid adhering to the magnetic beads 2 is usually very small, and therefore, the residual liquid rapidly volatilizes with the start of the heat generation treatment, and bumping occurs. The residual liquid spattered by the bumping is shaken off from the magnetic beads 2 together with other residual droplets other residual liquids, and is accumulated at the bottom of the container 1. The residual liquid thus accumulated can be collected again by the liquid discharging treatment. In this way, the amount of the residual elution liquid adhering to the magnetic beads 2 can be minimized. Accordingly, the yield can be further increased.

When the elution liquid is caused to bump, the temperature of the elution liquid associated with the heat generation treatment of the magnetic beads 2 may be set to be equal to or higher than the boiling point.

Only any one of the heat treatment of the elution liquid, the concentration treatment of the elution liquid, and the bumping treatment of the elution liquid included in the elution step S208 may be performed, or any two or more thereof may be performed.

### 2. Magnetic Bead

Next, the magnetic bead 2 will be described.

As described above, the magnetic bead 2 illustrated in FIG. 9 includes the magnetic metal particle 22 and the coating layer 24. The coating layer 24 preferably covers the entire surface of the magnetic metal particle 22, but may have a discontinuous portion. The coating layer 24 may be provided as necessary, or may be omitted.

### 2.1. Magnetic Metal Particle

The magnetic metal particles 22 are particles containing a magnetic metal. Examples of the constituent material of the magnetic metal particles 22 include an Fe-based alloy, a Co-based alloy, and a Ni-based alloy. In particular, from the viewpoint of obtaining high saturation magnetization, the constituent material of the magnetic metal particles 22 is preferably an Fe-based alloy (alloy containing Fe as a main component).

The Fe-based alloy may be an alloy having a content of Fe of 50% or more in terms of atomic ratio, and the content of Fe is preferably 70% or more.

The Fe-based alloy contains Fe as a main component, and may contain one or two or more selected from the group including Co, Ni, Cr, Nb, Cu, Al, Mn, Mo, Si, Sn, B, C, P, Ti, and Zr, depending on target characteristics.

An example of the Fe-based alloy is an alloy having a content of Si of preferably 1.0 atomic% or more and 30.0 atomic% or less, more preferably 1.5 atomic% or more and 13.0 atomic% or less, and still more preferably 2.0 atomic% or more and 7.0 atomic% or less. Since such an alloy has high permeability, the saturation magnetization tends to be high.

The Fe-based alloy may contain at least one of boron (B) having a content of 5.0 atomic% or more and 16.0 atomic% or less and carbon (C) having a content of 0.5 atomic% or more and 5.0 atomic% or less. These elements are elements that promote amorphization, and contribute to stably forming an amorphous structure or a nanocrystal structure in the magnetic metal particle 22.

The Fe-based alloy may contain Cr (chromium) having a content of 1.0 atomic% or more and 8.0 atomic% or less. Accordingly, corrosion resistance of the magnetic metal particle 22 can be improved.

A content of impurities is preferably 1.0 atomic% or less in total. At this level, the above effect exhibited by the magnetic metal particles 22 is less likely to be impaired even if impurities are contained. The impurity in the present specification is an element that is unintentionally mixed in raw materials of the magnetic metal particles 22 or during the production of the magnetic metal particles 22.

A main metal structure constituting the magnetic metal particle 22 may take various forms such as a crystal structure, an amorphous structure, and a nanocrystal structure. An amorphous structure refers to a non-crystal structure in which no crystal is present, and a nanocrystal refers to a structure mainly formed of fine crystals having a crystal grain diameter of 100 nm or less. The amorphous structure and the nanocrystal structure impart a high hardness to the magnetic metal particles 22. When the structure is the amorphous structure or the nanocrystal structure, the coercive force of the magnetic bead 2 has a particularly low value, which contributes to improvement in the redispersibility of the magnetic bead 2. A volume fraction of the magnetic metal particles 22 having an amorphous structure or a nanocrystal structure is preferably 40% or more, and more preferably 60% or more. The volume fraction is obtained based on a result of crystal structure analysis using X-ray diffraction. The magnetic metal particle 22 having such a structure has high magnetic permeability, and therefore, the magnetic metal particle 22 easily collects magnetic flux and can efficiently generate heat by induction heating.

The metal structure of the magnetic metal particle 22 can also be identified by performing crystal structure analysis using an X-ray diffraction method on the magnetic metal particle 22. Alternatively, the metal structure can be specified by analyzing a structure observation image or a diffraction pattern obtained with a transmission electron microscope (TEM) from a cut-out sample. For example, in the case of the amorphous structure, a diffraction peak derived from a metal crystal of an α-Fe phase or the like is not observed in peak analysis in the X-ray diffraction method. In the case of the amorphous structure, a so-called halo pattern is formed in an electron diffraction pattern using TEM, and formation of a spot due to a crystal is not observed. The nanocrystal structure is formed of a crystal structure having a grain diameter of, for example, 100 nm or less, and can be checked from a TEM observation image.

The magnetic metal contained in the magnetic metal particle 22 is particularly preferably an Fe-based amorphous alloy containing Fe, Cr, Si, and B. The Fe-based amorphous alloy is an Fe-based alloy including an amorphous structure. Such magnetic metal particles 22 have high magnetic permeability and a low coercive force. Therefore, the magnetic beads 2 having good separability in magnetic separation and excellent redispersibility can be obtained by using such magnetic metal particles 22. The magnetic metal particles 22 containing the Fe-based amorphous alloy have high magnetic permeability, and therefore, the magnetic metal particles 22 easily collect magnetic flux and can efficiently generate heat by induction heating.

### 2.2. Coating Layer

The coating layer 24 covers the magnetic metal particles 22 and contains gold or an inorganic oxide. According to such a configuration, a function of protecting the magnetic metal particles 22 can be imparted to the coating layer 24. Accordingly, elution of metal ions and the like from the magnetic metal particles 22 can be prevented. As a result, the occurrence of insufficient purification or insufficient inspection due to metal ions or the like can be prevented. The coating layer 24 may be provided as necessary, or may be omitted.

Examples of the inorganic oxide include a silicon oxide, a magnesium oxide, a calcium oxide, an aluminum oxide, a titanium oxide, a zirconium oxide, a boron oxide, an yttrium oxide, and a molybdenum oxide, and a mixture of one or two or more thereof may be used.

The inorganic oxide is preferably silicon oxide. Since the silicon oxide is chemically stable, oxidation and corrosion of the magnetic metal particles 22 can be particularly prevented, and the corrosion resistance of the magnetic beads 2 can be particularly improved. Good nucleic acid adsorptivity can be imparted to the coating layer 24.

The silicon oxide is represented by a composition formula of SiOₓ (0 < x ≤ 2), and is preferably SiO₂. The silicon oxide may form a composite oxide or a composite with one or two or more selected from the group including Al, Ti, V, Nb, Cr, Mn, Sn, and Zr.

The coating layer 24 may contain a substance (impurity) other than the inorganic oxide within a range in which an effect thereof is not impaired, for example, at a ratio of 50 mass% or less of the inorganic oxide described above. When the silicon oxide is used as the inorganic oxide, examples of the impurity include C, N, and P.

A composition of the inorganic oxide can be checked by, for example, EDX analysis or Auger electron spectroscopy.

The thickness of the coating layer 24 is preferably 10 nm or more and 200 nm or less, more preferably 15 nm or more and 100 nm or less, and still more preferably 20 nm or more and 100 nm or less. Accordingly, even when the magnetic beads 2 collide with each other or collide with an inner wall of the container 1 or the like, the coating layer 24 can be prevented from being broken or peeled off. As a result, elution of iron ions and the like due to exposure of the magnetic metal particles 22 can be prevented. A decrease in the magnetization per unit volume of the magnetic bead 2 can be prevented, and a decrease in the movement speed of the magnetic beads 2 can be prevented.

Since the gold has high corrosion resistance, a function of reducing oxidation and corrosion of the magnetic metal particles 22 is high. A decrease in the magnetization of the magnetic metal particles 22 can be prevented, and a decrease in the nucleic acid adsorption efficiency due to elution of iron ions or the like can be prevented by providing the coating layer 24 containing gold.

Gold may be present in a state of a simple substance, an alloy, or the like. When the coating layer 24 contains gold, the coating layer 24 may contain impurities other than gold.

The thickness of the gold-containing coating layer 24 is preferably 0.5 nm or more and 50 nm or less, more preferably 1 nm or more and 30 nm or less, and still more preferably 2 nm or more and 20 nm or less. Accordingly, the corrosion resistance of the magnetic bead 2 can be particularly favorably ensured. A decrease in the magnetization per unit volume of the magnetic bead 2 can be prevented, and a decrease in the movement speed of the magnetic beads 2 can be prevented.

The coating layer 24 may cover a surface of one magnetic metal particle 22, or may cover a plurality of magnetic metal particles 22 together.

The thickness of the coating layer 24 can be measured from, for example, a cross-sectional observation image of the magnetic bead 2 observed with a transmission electron microscope or a scanning electron microscope. Specifically, the thickness of the coating layer 24 can be calculated by acquiring a plurality of cross-sectional observation images of the coating layer 24 and averaging measured values from image processing or the like. For example, the thickness of the coating layer 24 is measured at five or more positions for one magnetic bead 2, an average value is determined, and then the average value is further averaged for ten or more magnetic beads 2.

### 2.3. Characteristics of Magnetic Bead

An average particle diameter D50 of the magnetic beads 2 is preferably 0.5 μm or more and 30 μm or less, more preferably 1 μm or more and 20 μm or less, still more preferably 2 μm or more and 15 μm or less, and particularly preferably 3 μm or more and 10 μm or less. When the average particle diameter D50 of the magnetic beads 2 is within the above-described range, a specific surface area of the magnetic beads 2 can be sufficiently large, and an attractive force and an adsorption force suitable for magnetic separation can be generated in the magnetic beads 2. In addition, aggregation of the magnetic beads 2 can be prevented, and dispersibility can be improved.

When the average particle diameter D50 of the magnetic beads 2 is less than the above-described lower limit, the value of the magnetization of the magnetic beads 2 is reduced, and the magnetic beads 2 are likely to aggregate. As a result, there is a concern that the adsorption efficiency of the nucleic acid is reduced, or the foreign substances are likely to be mixed. In addition, the movement speed of the magnetic bead 2 may decrease, and the time required for magnetic separation may increase. On the other hand, when the average particle diameter D50 of the magnetic beads 2 exceeds the upper limit, the specific surface area of the magnetic beads 2 is reduced, and therefore, a sufficient amount of the nucleic acid cannot be adsorbed, and a yield of the nucleic acid may decrease. In addition, the magnetic bead 2 is likely to settle, the magnetic bead 2 that can contribute to the extraction of the nucleic acid may decrease, and the yield of the nucleic acid may decrease.

The average particle diameter D50 of the magnetic beads 2 can be determined from a cumulative distribution curve obtained from a volume-based particle size distribution measured by a laser diffraction and dispersion method. Specifically, in the cumulative distribution curve, a particle diameter (median diameter) where a cumulative value is 50% from a small diameter side is the average particle diameter D50 of the magnetic beads 2. Examples of a device that measures the particle size distribution by using the laser diffraction/scattering method include MT3300 series manufactured by MicrotracBEL Corp.

A saturation magnetization of the magnetic metal particle 22 is preferably 50 emu/g or more, more preferably 80 emu/g or more, and still more preferably 100 emu/g or more. The saturation magnetization is a magnetization value in a case in which a magnetization exhibited by a magnetic material when a sufficiently large magnetic field is externally applied is constant regardless of the magnetic field. When the saturation magnetization is within the above range, a function as a magnetic material can be sufficiently exhibited. Specifically, since a movement speed of the magnetic bead 2 in a magnetic field can be increased, a time required for magnetic separation can be shortened. The saturation magnetization of the magnetic metal particle 22 affects an adsorption force when being fixed by an external magnetic field. When the saturation magnetization is within the above range, a sufficiently high adsorption force can be obtained, and therefore, when the liquid 3 is discharged in a state in which the magnetic bead 2 is immobilized, the magnetic bead 2 can be prevented from being discharged together with the liquid 3. Accordingly, a decrease in yield of nucleic acid due to a decrease in the magnetic beads 2 can be prevented.

An upper limit of the saturation magnetization of the magnetic metal particle 22 is not particularly limited, and is preferably 220 emu/g or less from the viewpoint of ease of selection of a material suitable for a balance between performance and cost.

The saturation magnetization of the magnetic metal particle 22 can be measured by a vibrating sample magnetometer (VSM) or the like. As the vibrating sample magnetometer, for example, TM-VSM1230-MHHL manufactured by Tamagawa Seisakusyo Co., Ltd. may be used. A maximum applied magnetic field when measuring the saturation magnetization is, for example, 0.5 T or more.

A coercive force Hc of the magnetic metal particle 22 is preferably 100 A/m or less, more preferably 80 A/m or less, still more preferably 60 A/m or less, and particularly preferably 50 A/m or less. The coercive force Hc refers to a value of an external magnetic field in an opposite orientation required to return a magnetized magnetic material to an unmagnetized state. That is, the coercive force Hc means a resistance force against an external magnetic field. As the coercive force Hc of the magnetic metal particle 22 decreases, the magnetic beads 2 are less likely to aggregate even when being switched from a state in which a magnetic field is applied to a state in which no magnetic field is applied, and the magnetic beads 2 can be uniformly dispersed in the liquid 3. Further, even when the switching of the magnetic field application is repeated, the redispersibility of the magnetic beads 2 can be improved. A lower limit of the coercive force Hc of the magnetic metal particle 22 is not particularly limited, and is preferably 5 A/m or more from the viewpoint of ease of selection of a material suitable for a balance between performance and cost.

The coercive force Hc of the magnetic metal particle 22 can be measured by a vibrating sample magnetometer or the like in the same manner as the saturation magnetization described above. A maximum applied magnetic field when measuring the coercive force Hc is, for example, 15 kOe.

### 2.4. Method for Producing Magnetic Bead

Next, an example of a method of producing the magnetic beads 2 will be described.

First, the magnetic metal particles 22 are prepared. The magnetic metal particles 22 are produced using a method according to a general metal powder production method. Examples of the production method include a melting process in which a metal is melted, solidified, and powdered, a chemical process in which a powder is produced by a reduction method or a carbonyl method, and a mechanical process in which a metal having a larger shape such as an ingot is mechanically pulverized to obtain a powder. Among them, a melting process such as an atomization method is suitable for producing the magnetic metal particles 22.

Next, the coating layer 24 is formed on the surface of the magnetic metal particle 22. Examples of the method of forming the coating layer 24 include wet formation methods such as a sol-gel method and a plating method, and dry formation methods such as a vacuum deposition method, a sputtering method, and an atomic layer deposition (ALD) method. Among them, a Stober method which is a type of sol-gel method or an ALD method is preferably used.

As described above, the magnetic bead 2 including the magnetic metal particle 22 and the coating layer 24 is obtained.

### 3. Effects of Embodiment

As described above, the biological material purification method according to the embodiment is a method for purifying a biological material, and includes the adsorption step S202, the separation step S204, and the elution step S208. In the adsorption step S202, the magnetic beads 2 and the biological material are mixed, and the biological material is adsorbed to the magnetic beads 2 in the obtained mixed liquid. In the separation step S204, the magnetic beads 2 to which the biological material is adsorbed are separated from the mixed liquid. In the elution step S208, the biological material is eluted from the magnetic bead 2.

The magnetic bead 2 contains a magnetic metal. A heat generation treatment of causing the magnetic beads 2 to generate heat by induction heating is included.

According to such a configuration, it is possible to provide a biological material purification method capable of efficiently purifying a biological material by causing the magnetic beads 2 to generate heat in a short time.

In the biological material purification method according to the embodiment, the mixed liquid may include a specimen sample in which a biological material is encapsulated in an outer shell. In this case, the adsorption step S202 may include a treatment of heating the mixed liquid by a heat generation treatment.

According to such a configuration, when the nucleic acid contained in the specimen sample is encapsulated in the outer shell, the nucleic acid can be efficiently extracted from the outer shell by heating the mixed liquid by the heat generation treatment.

The biological material purification method according to the embodiment may further include the washing step S206. In this case, in the washing step S206, the magnetic beads 2 to which the biological material is adsorbed and the wash solution are charged into a container, and cleaning is performed. The washing step S206 includes a treatment of immobilizing the magnetic beads 2 by a magnetic force (magnetic separation treatment), a treatment of discharging the wash solution from the container while immobilizing the magnetic beads 2 (liquid discharging treatment), and a treatment of vaporizing the wash solution by causing the immobilized magnetic beads 2 to generate heat by a heat generation treatment (drying treatment).

According to such a configuration, a small amount of the wash solution adhering to the magnetic beads 2 can be vaporized and removed in a short time. Accordingly, the wash solution (residual liquid) adhering to the magnetic beads 2 can be prevented from being brought into the elution step S208. As a result, when the nucleic acid after purification is tested, the occurrence of test defects associated with the residual liquid can be prevented.

In the biological material purification method according to the embodiment, the elution step S208 may include a treatment of bringing the magnetic beads 2 to which the biological material is adsorbed into contact with the elution liquid, and a treatment of concentrating the elution liquid by causing the magnetic beads 2 immersed in the elution liquid to generate heat by a heat generation treatment.

According to such a configuration, the concentration of the elution liquid can be increased, and the nucleic acid purification can be efficiently performed. Accordingly, when nucleic acid detection using, for example, real-time PCR is performed on the purified product, there is an advantage that fewer PCR cycles are required to reach a detectable level of an amplified product.

In the biological material purification method according to the embodiment, the elution step S208 may include a treatment of charging the magnetic beads 2 to which the biological material is adsorbed and the elution liquid in a container and then immobilizing the magnetic beads 2 by a magnetic force, a treatment of discharging the elution liquid from the container 1 while immobilizing the magnetic beads 2, and a treatment of causing the immobilized magnetic beads 2 to generate heat by a heat generation treatment and shaking off the elution liquid adhering to the magnetic beads 2 due to bumping of the elution liquid.

According to such a configuration, the amount of the residual elution liquid adhering to the magnetic beads 2 can be minimized. Accordingly, the yield can be further increased.

In the biological material purification method according to the embodiment, the magnetic metal is preferably an Fe-based amorphous alloy containing Fe, Cr, Si, and B.

According to such a configuration, when the Fe-based amorphous alloy is contained, the magnetic metal particle 22 has a high magnetic permeability and a low coercive force. Therefore, the magnetic bead 2 having good separability in magnetic separation and excellent redispersibility can be obtained.

In the biological material purification method according to the embodiment, the frequency of the induction heating is preferably 20 kHz or more and 100 kHz or less.

According to such a configuration, the depth of the skin effect can be optimized according to the particle diameter of the magnetic bead 2, and therefore, the heat generation treatment can be particularly efficiently performed.

The magnetic separation device 100 according to the embodiment includes the container holding unit 120 that holds the container 1 accommodating the magnetic beads 2 and extending along the axis AX coupling the bottom portion 12 and the opening portion 14, and the electromagnetic coil 92 disposed outside the side portion 16 coupling the bottom portion 12 and the opening portion 14.

According to such a configuration, the magnetic separation treatment can be performed while causing the magnetic beads 2 to generate heat in a short time, and therefore, the magnetic separation device 100 capable of efficiently purifying the biological material can be provided.

The magnetic separation device 100 according to the embodiment may further include the magnet 140 disposed on the side opposite to the side portion 16 via the electromagnetic coil 92.

According to such a configuration, even when the magnetic beads 2 are immobilized to the inner wall of the side portion 16, the magnetic beads 2 can efficiently generate heat. In addition, since the magnet 140 is disposed behind the electromagnetic coil 92, it is easy to immobilize the magnetic beads 2 in a vicinity of the electromagnetic coil 92. Therefore, the immobilized magnetic beads 2 can more efficiently generate heat.

Although the biological material purification method and the magnetic separation device according to the present disclosure have been described above based on the illustrated embodiments, the present disclosure is not limited thereto. For example, the biological material purification method according to the present disclosure may be a method in which a step for any purpose is added to the embodiment. In the magnetic separation device according to the present disclosure, any configuration may be added to the embodiment, or a part of the configuration of the embodiment may be replaced with another configuration having the same function.

### Examples

Next, specific examples of the present disclosure will be described.

### 4. Production of Magnetic Beads and Nucleic Acid Purification

Magnetic beads were produced as follows, and then nucleic acid purification was performed by a biological material purification method of each example and each comparative example using the prepared magnetic beads.

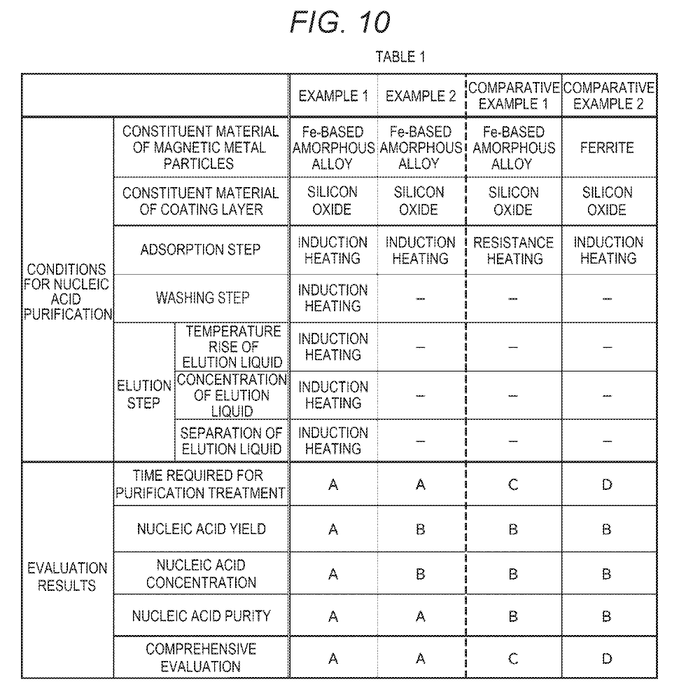

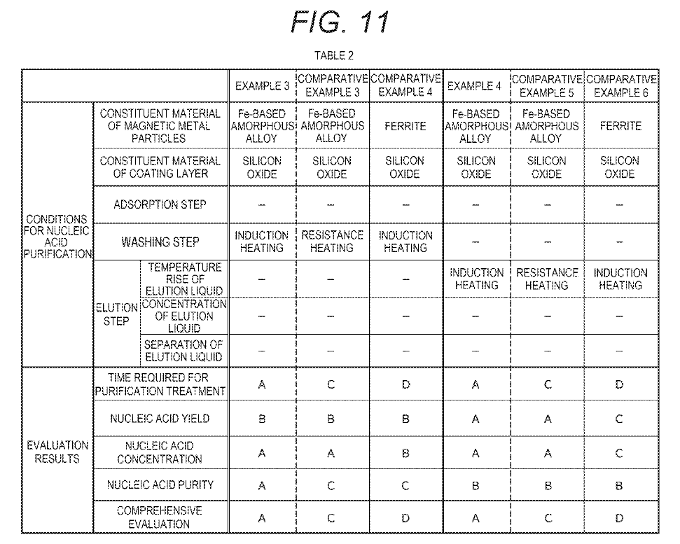

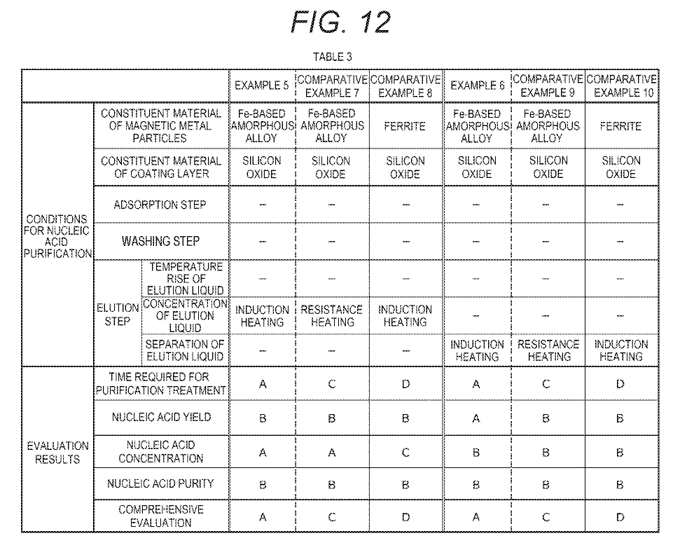

FIG. 10 is Table 1 illustrating conditions of nucleic acid purification and evaluation results of nucleic acid purification using the biological material purification methods of Examples and Comparative Examples. FIG. 11 is Table 2 illustrating conditions of nucleic acid purification and evaluation results of nucleic acid purification using the biological material purification method of Examples and Comparative Examples. FIG. 12 is Table 3 illustrating conditions of nucleic acid purification and evaluation results of nucleic acid purification using the biological material purification method of Examples and Comparative Examples.

5.1. Example 1

Nucleic acid purification using the biological material purification method of Example 1 will be described.

### 5.1.1. Production of Magnetic Bead

First, an Fe-based amorphous alloy powder produced by a water atomization method was prepared as magnetic metal particles.

Next, TEOS (tetraethoxysilane) was brought into contact with surfaces of the magnetic metal particles by a Stober method, and then firing was performed to form a coating layer formed of silicon oxide. Accordingly, magnetic beads were obtained.

### 5.1.2. Nucleic Acid Purification

First, a sample solution having a concentration of 2 [mg/mL] was prepared using genomic DNA and Buffer AVL buffer manufactured by Takara Bio Inc., and left to stand until the temperature reached room temperature.

The magnetic bead dispersion liquid containing the magnetic beads was taken into a 1.5 mL tube and left to stand until the room temperature was reached. The configuration of the magnetic beads used is as illustrated in Table 1 (FIG. 10).

Next, an adsorption step was performed. Specifically, another 1.5 mL tube was prepared, and 2 μL of the sample solution and 98 μL of artificial serum were dispensed. Next, 40 μL of a Proteinase K solution having a concentration of 1 mass%, 750 μL of a lysis-adsorption liquid, and a magnetic bead dispersion liquid were added to the tube. Then, the magnetic beads in the tube were caused to generate heat using an induction heating device to heat the substances accommodated in the tube. Subsequently, the substances accommodated in the tube were stirred with a vortex mixer for 10 minutes.

Next, a separation step was performed. Specifically, the tube after the stirring was set in a magnetic stand, and the magnetic separation treatment and the liquid discharging treatment were performed.

Next, a washing step was performed. Specifically, 900 μL of a wash solution adjusted to room temperature was added to the tube after the separation step. An aqueous solution containing 7.3 M of guanidine hydrochloride was used as the wash solution. Then, the substances accommodated in the tube were stirred for 5 seconds with a vortex mixer. Thereafter, a magnetic separation treatment was performed. In the magnetic separation treatment, the tube was set on a magnetic stand and left for 20 seconds. Subsequently, a liquid discharging treatment was performed to remove the wash solution in the supernatant. The above treatment was defined as a first cleaning treatment, and this treatment was performed again. Subsequently, 900 μL of a wash solution adjusted to room temperature was added to the tube after the first cleaning treatment. As the wash solution, 70 v/v% ethanol obtained by adding 70 mL of ethanol to 30 mL of water was used. Then, the substances accommodated in the tube were stirred for 5 seconds with a vortex mixer. Thereafter, a magnetic separation treatment was performed. In the magnetic separation treatment, the tube was set on a magnetic stand and left for 20 seconds. Subsequently, a liquid discharging treatment was performed to remove the wash solution in the supernatant. The above treatment was defined as a second cleaning treatment, and this treatment was performed again. Next, in a state in which the magnetic beads in the tube were immobilized using the magnetic separation device, the magnetic beads were caused to generate heat to dry the wash solution adhering to the magnetic beads.

Next, the elution step was performed. Specifically, 100 μL of an elution liquid adjusted to room temperature was added to the tube after the washing step. Pure water was used as the elution liquid. Then, the substances accommodated in the tube were stirred for 10 minutes with a vortex mixer. Next, the magnetic beads in the tube were heated using the induction heating device to heat the elution liquid and raise the temperature. Accordingly, elution of the nucleic acid captured by the magnetic beads was promoted. Subsequently, the elution liquid was further heated using the induction heating device. Accordingly, the elution liquid was concentrated. Thereafter, a magnetic separation treatment was performed. In the magnetic separation treatment, the tube was set on a magnetic stand and left for 30 seconds. Subsequently, a liquid discharging treatment was performed, and the elution liquid in the supernatant was collected. Next, in a state in which the magnetic beads in the tube were immobilized using the magnetic separation device, the magnetic beads were caused to generate heat, thereby bumping the elution liquid adhering to the magnetic beads. Accordingly, the elution liquid was shaken off and separated. The separated elution liquid was collected by performing the liquid discharging treatment again.

Table 1 shows the configuration of the magnetic beads used and the heating method in each step and each treatment in the step as the conditions of the nucleic acid purification.

The characteristics of the magnetic beads used are as follows.

Average particle diameter D50: 10 μm

Saturation magnetization: 110 emu/g

Coercive force: 50 A/m

Thickness of coating layer: 30 nm

The conditions for the induction heating are as follows.

Frequency of alternating current: 20 kHz

Output of electromagnetic coil: 100 W

### 5.2. Example 2 and Comparative Examples 1 and 2

In Example 2 and Comparative Examples 1 and 2, magnetic beads were prepared and nucleic acid purification was performed in the same manner as in Example 1 except that the configuration of the magnetic beads and the conditions for nucleic acid purification were changed as illustrated in Table 1 (FIG. 10).

Specifically, in Example 2, nucleic acid purification was performed in the same manner as in Example 1 except that the magnetic beads were caused to generate heat using the induction heating device in the adsorption step, and heating was omitted in the washing step and the elution step.

In Comparative Example 1, nucleic acid purification was performed in the same manner as in Example 2 except that resistance heating (heating of the accommodated substances by a heater) was performed instead of induction heating.

In Comparative Example 2, nucleic acid purification was performed in the same manner as in Example 2 except that ferrite particles were used instead of the magnetic metal particles. The term "ferrite" is a constituent material containing no magnetic metal.

### 5.3. Example 3 and Comparative Examples 3 and 4

In Example 3 and Comparative Examples 3 and 4, magnetic beads were produced and nucleic acid purification was performed in the same manner as in Example 1 except that the configuration of the magnetic beads and the conditions for nucleic acid purification were changed as illustrated in Table 2 (FIG. 11).

Specifically, in Example 3, nucleic acid purification was performed in the same manner as in Example 1 except that the magnetic beads were heated using the induction heating device in the washing step, and heating was omitted in the adsorption step and the elution step.

In Comparative Example 3, nucleic acid purification was performed in the same manner as in Example 3 except that resistance heating (heating of the accommodated substances by a heater) was performed instead of induction heating.

In Comparative Example 4, nucleic acid purification was performed in the same manner as in Example 3 except that ferrite particles were used instead of the magnetic metal particles.

### 5.4. Example 4 and Comparative Examples 5 and 6

In Example 4 and Comparative Examples 5 and 6, magnetic beads were produced and nucleic acid purification was performed in the same manner as in Example 1 except that the configuration of the magnetic beads and the conditions for nucleic acid purification were changed as illustrated in Table 2 (FIG. 11).

Specifically, in Example 4, nucleic acid purification was performed in the same manner as in Example 1 except that the magnetic beads were caused to generate heat using the induction heating device in the temperature raising treatment of the elution liquid in the elution step, and heating was omitted in the treatments remaining in the adsorption step, the washing step, and the elution step.

In Comparative Example 5, nucleic acid purification was performed in the same manner as in Example 4 except that resistance heating (heating of the accommodated substances by a heater) was performed instead of induction heating.

In Comparative Example 6, nucleic acid purification was performed in the same manner as in Example 4 except that ferrite particles were used instead of the magnetic metal particles.

### 5.5. Example 5 and Comparative Examples 7 and 8

In Example 5 and Comparative Examples 7 and 8, magnetic beads were prepared and nucleic acid purification was performed in the same manner as in Example 1 except that the configuration of the magnetic beads and the conditions for nucleic acid purification were changed as illustrated in Table 3 (FIG. 12).

Specifically, in Example 5, nucleic acid purification was performed in the same manner as in Example 1 except that the magnetic beads were heated using the induction heating device in the concentration treatment of the elution liquid in the elution step, and heating was omitted in the treatments remaining in the adsorption step, the washing step, and the elution step.

In Comparative Example 7, nucleic acid purification was performed in the same manner as in Example 5 except that resistance heating (heating of the accommodated substances by a heater) was performed instead of induction heating.

In Comparative Example 8, nucleic acid purification was performed in the same manner as in Example 5 except that ferrite particles were used instead of the magnetic metal particles.

### 5.6. Example 6 and Comparative Examples 9 and 10

In Example 6 and Comparative Examples 9 and 10, magnetic beads were prepared and nucleic acid purification was performed in the same manner as in Example 1 except that the configuration of the magnetic beads and the conditions for nucleic acid purification were changed as illustrated in Table 3 (FIG. 12).

Specifically, in Example 6, nucleic acid purification was performed in the same manner as in Example 1 except that the magnetic beads were heated using the induction heating device in the separation treatment of the elution liquid in the elution step, and heating was omitted in the treatments remaining in the adsorption step, the washing step, and the elution step.

In Comparative Example 9, nucleic acid purification was performed in the same manner as in Example 6 except that resistance heating (heating of the accommodated substances by a heater) was performed instead of induction heating.

In Comparative Example 10, nucleic acid purification was performed in the same manner as in Example 6 except that ferrite particles were used instead of the magnetic metal particles.

### 6. Evaluation of Nucleic Acid Purification

Next, results of nucleic acid purification in Examples and Comparative Examples were evaluated.

### 6.1. Time Required for Purification Treatment

In the nucleic acid purification in Examples and Comparative Examples, each time required for the step and treatment of performing heating was determined.

Specifically, in the adsorption step in which heating was performed, the time required for the temperature of the lysis-adsorption liquid in the vicinity of the magnetic beads to rise to a predetermined temperature was determined as the "required adsorption time".

In the washing step, the time until the drying of the wash solution adhering to the magnetic beads was completed was determined as "required drying time".

In the elution step, the time required for the temperature of the elution liquid to rise to a predetermined temperature was determined as the "time required for the temperature rise of the elution liquid".

In the elution step, the time required for the elution liquid to be concentrated to a predetermined concentration was determined as "time required for concentration of elution liquid".

In the elution step, the time required until it was determined that the elution liquid adhering to the magnetic beads was separated in appearance was determined as "time required for separation of elution liquid".

Next, the time required for the purification treatment was evaluated by observing each of the required times described above according to the following evaluation criteria. Evaluation results are illustrated in Tables 1 to 3.
A: each required time is shorter than 1 minute
B: each required time is 1 minute or longer and shorter than 3 minutes
C: each required time is 3 minutes or longer and shorter than 5 minutes
D: each required time is 5 minutes or longer

### 6.2. Nucleic Acid Yield

The nucleic acid purification in Examples and Comparative Examples was performed, the elution liquid was collected, and the amount of nucleic acid contained in the elution liquid (recovered nucleic acid amount) was measured. The method for measuring the nucleic acid yield is as follows.

First, the collected elution liquid was charged into a 1.5 mL tube, and 80 μL of the elution liquid was taken out therefrom and charged into a spectroscopic cell. Next, 240 μL of pure water was added to the spectroscopic cell to prepare a sample.

Next, the spectroscopic cell containing the sample was set in a cell holder of a spectrophotometer. As the spectrophotometer, a NanoDrop microspectrophotometer manufactured by Thermo Fisher Scientific was used. Then, a concentration of the nucleic acid in the elution liquid was measured with a spectrophotometer.

Next, the recovered nucleic acid amount was calculated using the measured nucleic acid concentration and the liquid amount of the elution liquid.

Next, the nucleic acid yield (recovery rate) was calculated using the recovered nucleic acid amount and the input nucleic acid weight.

The calculated nucleic acid yield was evaluated in view of the following evaluation criteria. Evaluation results are illustrated in Tables 1 to 3.
A: the yield of nucleic acid is 60% or more
B: the yield of nucleic acid is 50% or more and less than 60%
C: the nucleic acid yield is less than 50%

6.3. Nucleic Acid Concentration

The nucleic acid concentration obtained in the process of calculating the above nucleic acid yield was relatively evaluated in view of the following evaluation criteria. Evaluation results are illustrated in Tables 1 to 3.
A: the nucleic acid concentration is particularly high, and the test can be sufficiently efficient
B: the nucleic acid concentration is slightly high, and the test can be performed more or less efficiently
C: the nucleic acid concentration is low, and the efficiency of the test cannot be improved

6.4. Nucleic Acid Purity

First, the spectroscopic cell containing the collected elution liquid was set in a spectrophotometer, and the absorbances at wavelengths of 260 nm and 280 nm were measured. The nucleic acid base has an absorption maximum near 260 nm, and therefore, the absorbance at a wavelength of 260 nm represents the concentration of the nucleic acid. Proteins that can be foreign substances have an absorption maximum near 280 nm, and therefore, the absorbance at a wavelength of 280 nm represents the concentration of foreign substances.

Next, a ratio A260/A280 of the absorbance A260 to the absorbance A280 was calculated. Then, the nucleic acid purity was evaluated by comparing the calculation result with the following evaluation criteria. Evaluation results are illustrated in Tables 1 to 3.
A: the ratio A260/A280 is 2.1 or more
B: the ratio A260/A280 is 2.0 or more and less than 2.1
C: the ratio A260/A280 is less than 2.0

6.5. Comprehensive Evaluation

The comprehensive evaluation of nucleic acid purification was performed by observing the results of the above four evaluations according to the following evaluation criteria. Evaluation results are illustrated in Tables 1 to 3.
A: the number of evaluation values B is two or less, and the remaining evaluation values are all evaluation values A
B: the number of evaluation values B is three or more, and the remaining evaluation values are all evaluation values A
C: one or more evaluation values C are included
D: one or more evaluation values D are included

From the evaluation results illustrated in Tables 1 to 3, the following can be recognized.

In the nucleic acid purification of Examples, when the heat generation treatment of the magnetic beads by induction heating is included, the time required for the purification treatment could be shortened, and efficiency could be improved.

When the magnetic beads do not contain a magnetic metal (when the magnetic beads are formed of ferrite) or when resistance heating is used, it is not possible to sufficiently achieve efficiency.

What is claimed is:

1. A biological material purification method, which is a method for purifying a biological material, the method comprising:
an adsorption step of mixing magnetic beads and the biological material and adsorbing the biological material to the magnetic beads in the obtained mixed liquid;
a separation step of separating, from the mixed liquid, the magnetic beads to which the biological material is adsorbed; and
an elution step of eluting the biological material from the magnetic beads, wherein
the magnetic beads contain a magnetic metal, and
a heat generation treatment of causing the magnetic beads to generate heat by induction heating is performed.

2. The biological material purification method according to claim 1, wherein
the mixed liquid contains a specimen sample in which the biological material is encapsulated in an outer shell, and
the adsorption step includes a treatment of heating the mixed liquid by the heat generation treatment.

3. The biological material purification method according to claim 1, further comprising:
a washing step of cleaning the magnetic beads to which the biological material is adsorbed by charging the magnetic beads and a wash solution into a container, wherein
the washing step includes
a treatment of immobilizing the magnetic beads by a magnetic force,
a treatment of discharging the wash solution from the container while immobilizing the magnetic beads, and
a treatment of vaporizing the wash solution by heating the immobilized magnetic beads by the heat generation treatment.

4. The biological material purification method according to claim 1, wherein
the elution step includes
a treatment of bringing the magnetic beads to which the biological material is adsorbed into contact with an elution liquid, and
a treatment of concentrating the elution liquid by causing the magnetic beads immersed in the elution liquid to generate heat by the heat generation treatment.

5. The biological material purification method according to claim 1, wherein
the elution step includes
a treatment of immobilizing, after charging the magnetic beads to which the biological material is adsorbed and an elution liquid into a container, the magnetic beads by a magnetic force,
a treatment of discharging the elution liquid from the container while immobilizing the magnetic beads; and
a treatment of causing the immobilized magnetic beads to generate heat by the heat generation treatment and shaking off the elution liquid adhering to the magnetic beads by bumping of the elution liquid.

6. The biological material purification method according to claim 1, wherein
the magnetic metal is an Fe-based amorphous alloy containing Fe, Cr, Si, and B.

7. The biological material purification method according to claim 1, wherein
a frequency of the induction heating is 20 kHz or more and 100 kHz or less.

8. A magnetic separation device comprising:
a container holding unit configured to hold a container that accommodates magnetic beads and that extends along an axis coupling a bottom portion and an opening portion; and
an electromagnetic coil disposed outside a side portion coupling the bottom portion and the opening portion.

9. The magnetic separation device according to claim 8, further comprising:
a magnet disposed on a side opposite to the side portion via the electromagnetic coil.
